(19) United States
(12) Patent Application Publication
(10) Pub. No.: US 2025/0339619 A1
(43) Pub. Date: Nov. 6, 2025

(54) AUTO-INJECTOR SYSTEM

(71) Applicant: Meridian Medical Technologies, LLC, Columbia, MD (US)

(72) Inventor: Girum Yemane-Tekeste, Gaithersburg, MD (US)

(21) Appl. No.: 18/854,401

(22) PCT Filed: Apr. 4, 2023
(86) PCT No.: PCT/US2023/017364

§ 371 Date: Oct. 4, 2024

Related U.S. Application Data
(60) Provisional application No. 63/362,597, filed on Apr. 6, 2022.

(51) Int. Cl. A61M 5/20; A61J 1/20; A61M 5/315; A61M 5/32

(52) U.S. Cl. CPC A61M 5/2033; A61J 1/2048; A61M 5/3158; A61M 5/3202

(57) ABSTRACT

Systems and methods for automatically reconstituting a drug and then administering it to a patient are provided. The system and methods may include a reconstitution auto-injector having a first chamber having a powder contained therein, a second chamber having a liquid contained therein, a first power source, and a second power source. The first power source may be activated to compress the liquid to flow into the first chamber with the powder. The second power source may activate to inject the reconstituted drug into the patient.

### PRIORITY

The instant application claims priority to U.S. Provisional Application No. 63/262,597, filed Apr. 6, 2022, which is incorporated herein in its entirety.

### BACKGROUND

Lyophilized drugs and vaccines are commercially limited because of the complexity of reconstituting the drug into a useable form. Conventionally, a lyophilized drug is reconstituted from a vial using equipment and a number of steps. Once reconstituted, the drug is delivered to a patient through IV infusion, intramuscular or subcutaneous injections.

### SUMMARY

Exemplary embodiments of the systems and methods described herein are designed to reconstitute a drug and administer it automatically to a patient. The system stores a powder material, such as a drug (lyophilized) and its diluent in two separate chambers. During administration, the system and methods are configured to reconstitute the drug by automatically mixing the diluent with the powder, and thereafter inject the reconstituted material into a recipient. As used herein, drug is understood to include an material used for the health of a person. A drug may include a medication for responding to illnesses and/or symptoms, and/or in preventing such illnesses and/or symptoms. Although embodiments are described herein in terms of a powdered drug and a liquid used for a diluent, other combinations of mixed materials may also be used according to embodiments described herein. For example, two liquids may be combined, two powders may be combined, a powder and a combination liquid, etc.

Exemplary embodiments described herein include a reconstitution auto-injector and/or system of using the reconstitution auto-injector in order to administer a drug to a patient. Exemplary embodiments of the reconstitution auto-injector may include a first chamber configured to store a powder therein; and a second chamber configured to store a liquid. Although described herein in terms of the first chamber configured to store and/or in storing a powder, the first chamber may store a liquid. Although described herein in terms of the second chamber configured to store and/or in storing a liquid, the second chamber may store a powder. The reference to a first and/or second is intended to identify one chamber from another, and is not intended to specify a specific number of chambers or in defining an order of chambers. Therefore, the first chamber may be the second chamber or visa verse when describing different exemplary embodiments of the invention. The first chamber and the second chamber may be positioned side by side and separated by a separator.

The reconstitution auto-injector may include a body. The body may comprise an outer body at a first end and an inner body at a second end. The second end may include an end of the system configured to be positioned adjacent an injection site during use. A portion of the inner body may be positioned within a portion of the outer body. The outer body may move independently of the inner body. The inner body may slide longitudinally within the outer body. The configuration and/or positions of the inner body and outer body may provide for the automatic actuation of the system to recombine the drug and administer the drug to the patient through injection.

In an exemplary embodiment, the first chamber may be configured to retain and/or may retain a powder positioned therein, and the second chamber may be configured to retain and/or may retain a liquid positioned therein. The separator between the first chamber and the second chamber may include a flow channel providing fluid communication between the first chamber and the second chamber. The reconstitution auto-injector may also include a stopper positioned within the second chamber to obstruct the flow channel so that the first chamber and the second chamber are not in fluid communication in a first configuration or position of the stopper. The configuration of the chambers and/or stopper may permit the separation of the drug and its diluent for long term storage. The configuration of the chambers and/or stopper and/or flow channel are configured to permit the reconstitution of the drug and the diluent upon activation of the system to more easily administer a combination drug to a patient. Exemplary embodiments may also use the liquid and/or a gas to assist in the reconstitution of the drug by mixing the powder and the liquid.

The reconstitution auto-injector may include a first power source and a second power source. The first power source in communication with a first plunger and configured to move the plunger away from the first power source, and the second power source in communication with a second plunger and configured to move the plunger away from the second power source. As described herein, a power source is understood not to just include electrical power but mechanical power, such as movement and/or actuation of the system components for automatic constitution and/or automatic injection of the reconstituted drug.

The second power source may be activated by pressure on the inner body as the system is pressed against an injection site of a patient. The second power source may include a first compressed spring configured to release upon activation and extend to move a second plunger rod and the second plunger away from the second power source and compress a volume of the second chamber comprising the liquid.

The first power source may include a second compressed spring configured to release upon activation and extend to move the first plunger rod and the first plunger away from the first power source and compress a volume of the first chamber.

The reconstitution autoinjector may include an actuator positioned between the first plunger and the first power source to release the first spring as the first plunger is moved toward the first power source.

The reconstitution auto-injector may also include a needle, a stopper, and (optionally) an end cap. The stopper may be positioned within the first chamber adjacent the end cap. The needle may extend into the stopper and may move through the stopper and end cap when a plunger pushes again a terminal end of the needle. The needle may have a pointed end on an opposite end of the need from the terminal end pushed on by the plunger.

The reconstitution auto-injector may optionally include a safety cap.

Exemplary embodiments described herein, include a method of using a reconstitution auto-injector. The method may include providing the reconstitution auto-injector; activating the reconstitution auto-injector; reconstituting a drug in a powder form into a reconstituted drug mixture; and injecting the reconstituted drug mixture into a patient.

The reconstitution auto-injector used in the method may have any combination of the components described herein. For example, the reconstitution auto-injector may have a powder contained in a first chamber and a liquid contained in a second chamber. The powder and liquid may be separated from each other in a first configuration prior to use.

Exemplary embodiments of the method described herein may include activating the reconstitution auto-injector by pressing the reconstitution auto-injector against a skin of the patient to automatically trigger the reconstitution and the injection.

The method may include, during the activation, releasing a second compressed spring to move a second plunger in a direction away from the second compressed spring and toward an injection site.

The method may include opening a flow channel between the second chamber and the first chamber so that as the second plunger moves by the second spring, the liquid moves into the first chamber.

The method may include applying a gas through the flow channel after the liquid from the second chamber moves into the first chamber to agitate the liquid and reconstitute the powder into the liquid creating a reconstituted mixture, and blocking the flow channel with the second plunger.

The method may include actuating a first compressed spring by moving a first plunger in the first chamber as the first chamber is filled with the liquid from the second chamber, thereby releasing the first compressed spring and applying a force on the first plunger to move the first plunger away from the first spring and toward the injection site.

The method may include moving a needle within the first chamber with the first plunger through a stopper and out an end cap.

The method may include dispelling gas contained within the first chamber through the needle after an end of the needle exits the stopper, while the end of the needle is within a gap created by the end cap and before the end of the needle enters a skin of a patient.

The method may include moving the needle into the patient at the injection site while dispensing the reconstituted mixture into the patient.

Exemplary embodiments described herein may include a reconstitution auto-injector, having a body including a first chamber configured to store a powder therein; a first plunger within the first chamber; a first power source configured to push the first plunger and reduce a volume within the first chamber; a first actuator configured to trigger the first power source; a second chamber configured to store a liquid; a second plunger in the second chamber; and a second power source configured to push the second plunger and reduce a volume within the second chamber; a flow passage between the first chamber and the second chamber such that the liquid can flow into the first chamber when the second plunger is moved by the second power source. The first plunger, first actuator, and first power source may be configured such that the activator automatically triggers the first power source when the liquid of the second chamber flows into the first chamber and moves the plunger in a first direction to increase the volume of the first chamber. Once triggered, the first power source may be configured to push the first plunger in an opposite direction to reduce the volume within the first chamber.

The reconstitution auto-injector may include a stopper configured to cover the flow passage in a first configuration, and uncover the flow passage in a second configuration. The second configuration may be configured to occur when the liquid in the second container is compressed by the second plunger after activation of the second power source.

Exemplary embodiments of the methods of using a reconstitution auto-injector described herein may include providing the reconstitution auto-injector having a first chamber having a powder contained therein, a second chamber having a liquid contained therein, a first power source, and a second power source; activating the second power source to start the first power source and compress the liquid and flowing the liquid from the second chamber to the first chamber; reconstituting a drug in a powder form into a reconstituted drug mixture; and automatically activating the first power source to inject the reconstituted drug mixture into a patient.

The method may include the automatic activation of the first power source is through a movement of a first plunger in a first direction, and the first power source is configured to move the first plunger, once activated, in a second direction opposite the first direction.

The method may include the activation of the second power source is by pressing the reconstitution auto-injector against a skin of a patient to activate the second power source.

The method may include keeping the first chamber and second chamber out of fluid contact through a stopper over a flow channel, and then permitting fluid flow from the second chamber to the first chamber by moving the stopper from the flow channel.

### DRAWINGS

FIG. 1 illustrates a perspective view of an exemplary auto-injector according to embodiments of the invention.

FIG. 2A illustrates a side view of the exemplary auto-injector according to embodiments of the invention. FIG. 2B illustrates a cross sectional view to illustrate internal component parts of the system according to embodiments of the invention.

FIGS. 3A-3B illustrate exemplary features of the auto-injector according to embodiments of the invention including a safety feature and the auto-injector during a safety release portion of the method for administering a drug using an auto-injector according to embodiments described herein.

FIG. 4 illustrates a cross sectional view of the auto-injector during an activation portion of the method for administering a drug using an auto-injector according to embodiments described herein.

FIGS. 5-7 illustrates a cross sectional view of the auto-injector during a reconstitution portion of the method for administering a drug using an auto-injector according to embodiments described herein.

FIGS. 8-9 illustrates a cross sectional view of the auto-injector during an injection portion of the method for administering a drug using an auto-injector according to embodiments described herein.

FIG. 10 illustrates a cross sectional view of a portion of the auto-injector after completing the method for administering the drug using the auto-injector.

### DESCRIPTION

The following detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention. It should be understood that the drawings are diagrammatic and schematic representations of exemplary embodiments of the invention, and are not limiting of the present invention nor are they necessarily drawn to scale.

Exemplary embodiments of the system described herein may include one or more benefits. For example, exemplary embodiments of the systems and methods described herein may meet the Federal Drug Administration's (FDA's) contemporary emergency-use devices' reliability requirements. The systems and methods described herein may reconstituted a drug into a mixture prior to the injection process. The system, such as through the use of one or more automatic power source(s), may be configured to automate the reconstitution and/or injection processor so that user interfaces or steps are minimized. Device interfaces may be used that are compatible with current device, and/or may be configured to permit the addition of a needle shield and/or cover. Exemplary embodiments may have the liquid comprise a second liquid state drug (or combination of additional drugs) to administer a two or more drug combination. Exemplary embodiments may include a safety feature such as a safe pin at the front of the device.

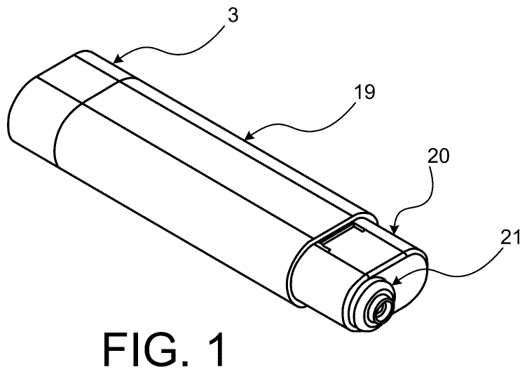

FIG. 1 illustrates a perspective view of an exemplary reconstitution auto-injector according to embodiments of the invention.

Exemplary embodiments of the reconstitution auto-injector described herein includes a first chamber configured to store a powder therein, and a second chamber configured to store a liquid. The chambers may be contained within a body. The first chamber and the second chamber may be within the inner body 20 and positioned side by side and separated by a separator.

FIG. 1 illustrates an exemplary exterior body that may be configured to enclose and/or define the fist chamber and the second chamber. As illustrated, the body may include an outer body 19 at a first end and an inner body 20 at a second end of the reconstitution auto-injection. The second end of the reconstitution auto-injector includes an end cap 21 at the end of the inner body 20 configured to be positioned adjacent an injection site during use. A portion of the inner body 20 may be positioned within a portion of the outer body.

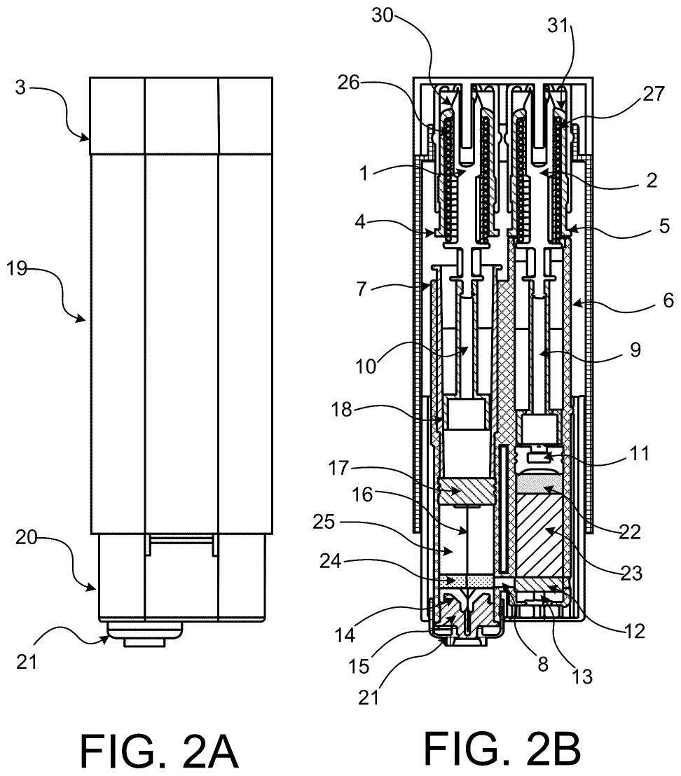

FIG. 2A illustrates a side view of the exemplary reconstitution auto-injector according to embodiments of the invention. FIG. 2B illustrates a cross sectional view of FIG. 2A to illustrate internal component parts of the system according to embodiments of the invention.

As illustrated, the exemplary reconstitution auto-injector may include an outer body 19 and an inner body 20. A portion of the inner body 20 may be positioned within a portion of the outer body 19 and extend out an end of the outer body 19. The inner body 20 may be at an end of the reconstitution auto-injector to define an end to be positioned at the injection site.

The inner body 20 and outer body 19 may define a first interior space 7 and a second interior space 6. The first interior space 7 may have positioned therein a first plunger 17 and the second interior space may have positioned therein a second plunger 11. The area within the first interior space from the first plunger to an end of the interior space toward the injection site (and away from the first power source) may define a first chamber. The area within the second interior space from the second plunger to an end of the interior space toward the injection site (and away from the second power source) may define a second chamber. The first chamber may be configured to retain and may have retained therein a powder 24. The second chamber may be configured to retain and may have retained therein a liquid 23. The second chamber may be subdivided into one or more sections, such as a first section configured to retain the liquid 23 therein and a second section configured to retain a gas 22 therein. The first section and second section may be in fluid communication and divided simply by the separation of the gas from the liquid and the positioning of the system such that the liquid is positioned toward the end closest to the injection site, and the gas is positioned away from the end. The first section and the second section may also include a barrier or other separation between the first section and the second section. The barrier may be configured to move within the inner body such that the second plunger may be configured to compress a gas 22 within the second section to push on the divider and then compress a liquid (such as a diluent) 23 within the second section.

In an exemplary embodiment of the reconstitution auto-injector, the first plunger 17 and the second plunger 11 are positioned within the inner body 20 to define the first chamber and the second chamber. The first chamber and the second chamber may be positioned side by side toward an end of the system configured to be positioned adjacent the injection site (and opposite from an end of the system comprising the first and/or second power source(s)). The inner body may comprise a separator between the first chamber and the second chamber. The separator may comprise a flow channel 8 permitting fluid flow between the first chamber and the second chamber through the flow channel. The second chamber may have positioned therein a stopper 12 coupled to a plug 13. The stopper may be positioned to obstruct the flow channel 8 in a first position and permit fluid flow through the flow channel 8 in a second position. In an exemplary embodiment, the second position is when the stopper is moved toward the end of the system configured to be positioned adjacent the injection site, compressing or moving along the plug 13. The first chamber and second chamber may therefore be separated and not in fluid communication when the stopper is in the first position and may be in fluid communication through the flow channel when the stopper is in the second position.

In an exemplary embodiment of the reconstitution auto-injector, the inner body 20 may include an end cap 21. The end cap 21 may be on a same side of the body as the first chamber and may be positioned at the end of the chamber toward the end of the system to be positioned adjacent to the injection site during use. The inner body 20 may comprise an opening such that the end of the first chamber is open through the inner body. The inner body may have a stopper 15 and end cap 21 to close the opening of the inner body and enclose the first chamber.

In an exemplary embodiment of the reconstitution auto-injector, a needle 16 may have a sharp end for penetrating an injection site. The sharp end may be positioned toward the end of the reconstitution auto-injector system configured to be positioned adjacent the injection site during use. As illustrated, the sharp end of the needle 16 may be positioned within a portion of the stopper 15. The opposite end of the needle is configured to abut the first plunger 17 and not penetrate the plunger. The first plunger 17 may therefore be configured to move within the first chamber, apply force to the needle (from the power source as described herein), and move the needle through the stopper 15 and end cap 21. The first plunger 17 may be positioned within the inner body 20 and the first interior space 7 to move within the first chamber toward and away from the needle according to the method described herein. For example, the first plunger 17 may have an exterior profile that is approximately equal to or minutely smaller than an interior profile of the first interior space and/or first chamber so that the first plunger 17 may slide within the first interior space 7 and the first chamber.

In an exemplary embodiment of the reconstitution auto-injector, the end cap 21 may be configured to separate the end of the needle after it leaves the stopper 15 and before the needle is injected into an injection site of a patient. The end cap 21 may include a gap to create the separation. The separation may permit the plunger 17 to move within the first chamber for a sufficient distance to dispel captured gas therein through the needle before the needle penetrates the patient at the injection site and the reconstituted drug mixture is administered to a patient.

As illustrated, the first chamber includes gas 25 and powder 24. The second chamber includes gas 22 and liquid 23. The gas may be any gas, such as air. The exemplary gas may be inert to the administered drug or other component parts such as the powder or liquid. In an exemplary embodiment of the reconstitution auto-injector, the powder comprises a drug. The drug may be freeze dried or in other powdered form. For example, the drug may comprise a lyophilized solid state drug. The liquid may be a diluent used to reconstitute the drug. In an exemplary embodiment, the liquid may comprise a second drug or other combination of drugs, vitamins, minerals, activators, or other composition to assist in the administration of the drug to the patient. Although described herein in terms of a drug, the powder may be any powdered material intended to be reconstituted and injected into a site. Although described herein, the injection of a drug is described with respect to a patient. However, any injection site for injection of a reconstituted substance may be considered within the scope of the present disclosure. For example, animals or other mammals may also benefit from exemplary embodiments described herein.

As illustrated, the exemplary auto-injector may comprise a first power source 1, and a second power source 2. The first power source 1 may include a first inner tube 4 having positioned therein a first spring 26 in communication with a first plunger rod 10 connected to a first plunger 17 in order to move a first plunger 17 through a first chamber volume and toward an end of the reconstitution auto-injector system configured to be positioned at an injection site. The second power source 2 may include a second inner tube 5 having positioned therein a second spring 27 in communication with a second plunger rod 9 connected to a second plunger 11 in order to move the second plunger through a second chamber volume and toward the end of the system configured to be positioned at the injection site.

The first power source 1 and the second power source 2 may each be configured in a first position in which the first spring 26 and second spring 27 are compressed and the first plunger rod 10 and the second plunger rod 9 are in retracted positions (positioned away from the injection site). Once activated, the spring 26, 27 is released so that the spring extends and applies a force through the rod 10, 9 and moves the plunger 17, 11 toward the end of the system positioned at the injection site, thereby compressing the space of the chamber 7, 6. The first power source and the second power source are configured to operate sequentially and automatically, such that that after the second power source is actuated, the second spring is released to elongate the spring, move the second plunger, actuate the first power source, releasing the first spring to elongate the first spring and move the first plunger toward the needle.

In an exemplary embodiment of the reconstitution auto-injector, the auto-injector may also include an activator 18. The activator may be positioned between the first plunger 17 and toward first power source 1. The activator may move within the first interior space 7 and pushed toward the first power source 1 as the first chamber is filled by the liquid 23 during the method described herein. The activator may release the first spring to activate the first power source 1. When the first power source 1 is activated, the first spring 26 may be released and the first spring elongates thereby applying a force through the rod 10 to move the rod toward the plunger 17, toward the needle 16, to move the needle through the stopper 15, out the end cap 21, and into the patient at the injection site.

In an exemplary embodiment of the reconstitution auto-injector, each of the power sources comprises a spring 26, 27 that can be held in their compressed state by a collet 30, 31 and inner tube interface 4, 5. When the inner tube 4, 5 is pushed, it permits the opposite end of the inner tube 4,5 to expand and/or the collet 30, 31 to be squeeze/compressed so that the collet 30, 31 may move past the retention and release the springs 26, 27.

In an exemplary embodiment of the reconstitution auto-injector, the auto-injector may automatically actuate the second power source 2 during use. For example, the inner body 20 may be configured at its end, such as through the end cap 21, to contact the patient and be positioned adjacent the injection site. The outer body 19 may be held by a practitioner injecting the drug mixture into the patient. As the inner body 20 and/or end cap 21 contacts the patient and a force is applied between the inner body and the outer body, the second power source 2 is actuated, thereby releasing the second spring 27.

In an exemplary embodiment of the reconstitution auto-injector, plunger 17 moves in the direction of first power source 1 as fluid is being transferred from the second chamber to the first chamber. The fluid applies pressure on plunger 17 displacing it to press on to the activator 18. The activator 18 can then press on to the first power source's inner tube 4 releasing the first power source's spring 26.

As illustrated, the system may comprise a safety cap 3 (also referred to herein as a safety pin). The safety cap 3 may be positioned on a second end of the system away from the end that is configured to be positioned at the injection site. The safety cap 3 may cover the first and/or second power sources to minimize the inadvertent activation of the system until the auto-injector is ready for use. In an exemplary embodiment of the reconstitution auto-injector, the safety cap 3 may be positioned about the second end of the system and cover an exposed portion of the first power source and an exposed portion of the second power source. The safety cap may be optional.

In an exemplary embodiment, the system may also include a disc 14. The disc 14 may be positioned adjacent the stopper 15. The disc 14 may be positioned between the stopper 15 and the flow channel 8 and define a terminal end of the first chamber in which the drug (whether in powder or recombined in a liquid) is configured to contact. The disc 14 may therefore provide a barrier between the drug and the stopper.

As illustrated, each of the power sources, comprise a spring positioned within a tunnel. The spring is illustrated as a coil spring positioned about a shaft, wherein the shaft couples to the plunger rod. The shaft comprises a flange positioned along the length of the shaft and extending radially outward from the shaft and circumferentially about the shaft. The flange is configured to contact a terminal end of the spring. The spring may therefore elongate and apply a force to the flange and thereby move the plugger rod. The plunger rod may thereafter contact the plunger to move the plunger to compress the volume of the chamber. Exemplary embodiments may include other configurations of the plunger, plunger rod, actuator, and force mechanism (such as a spring) that may be configured to operate in the same way. For example, different spring configurations may be used, and/or the spring mechanism may be integrated into the shaft, such as by a selection of a compressible/expandable material.

In an exemplary method using a reconstitution auto-injector system as described herein, a drug is reconstituted and administered automatically. In an exemplary embodiment, the method may comprise any combination of steps as described herein. In an exemplary embodiment, the an auto-injector is provided including a first chamber having a powder and a second chamber having a liquid. The method may include activating a first power source to apply pressure to the liquid and opening a fluid flow channel between the first chamber and the second chamber so that the liquid is transferred under pressure from the second chamber into the first chamber. The method includes applying gas to the first chamber through the flow channel to agitate the powder and the liquid to create a reconstituted mixture. The method may include activating a second power source to apply pressure to a plunger of the first chamber. The second power source is configured to move the needle. The second power source continues to apply force to the needle to move the needle. The method may include continuing to move the needle by the second power source through a stopper to exhaust the gas from the first chamber. The method may continue by moving the needle toward and into the injection site. The needle may access the reconstituted mixture and permit the reconstituted mixture to be administered through the needle and into the injection site.

First, a reconstitution auto-injector may be provided according to embodiments described herein. The auto-injector may comprise a two chamber system in which a first chamber is configured to store a powder, such as a lyophilized solid state drug, and a second chamber configured to store a liquid diluent. The auto-injector may also comprise a first power source configured to mix the powder with the diluent, and a second power source configured to administer the reconstituted drug.

Next, in an exemplary embodiment, the first power source is activated when the nose of the device is pressed against an injection site, which may cause the injector body of the auto-injector to push against the first power source's inner tube and release a spring of the first power source. When the spring of the first power source releases, it may push against the diluent chamber gas, diluent, and the diluent chamber stopper, and open a flow channel to the powder chamber. With the flow channel open, the diluent may flow under pressure from the second chamber to the first chamber to reconstitute the powder and the diluent to create a reconstituted drug.

After the diluent is transferred into the first chamber, the second chamber's gas is transferred through the flow channel bubbling gas into the reconstituted drug thereby bubbling gas into the reconstituted drug and agitating it to increase reconstitution and create a reconstituted drug mixture.

In an exemplary embodiment, the first power source continued to apply pressure and compress the gas in the first chamber, while moving to its final position and closing the flow channel. The compressed gas may apply a load on the first chamber's plunger and displace the plunger in the direction of the second power source. The plunger of the first chamber, after a duration, may push an activator, which can push on the second power source's inner tube and release a second spring of the second power source.

In an exemplary embodiment, the spring of the second power source may release and push against the first chamber's plunger in a direction of the injection site compressing the gas in the first chamber. The spring of the second power source may continue to apply force on the first chamber plunger, which may cause the first chamber plunger to push against a needle.

In an exemplary embodiment, the spring of the second power source may continue to apply force on the first chamber plunger, displacing the needle by the first chamber plunger penetrating the first chamber stopper and exhausting the compressed air through the needle.

In an exemplary embodiment, the spring of the second power source may continue to apply force on the first chamber plunger, thereby causing the needle to continue to move in the direction of the injection site, and thereafter, the needle is inserted into the injection site.

In an exemplary embodiment, the spring of the second power source continues to apply force on the first chamber plunger, the needle accesses the reconstituted drug mixture, and the reconstituted drug mixture flows through the needle orifice and into the injection site.

In an exemplary embodiment, the spring of the second power source continued to apply force on the first chamber plunger, the needle continues to deploy in the injection site, while simultaneously injecting the drug.

In an exemplary embodiment, the spring of the second power source continues to apply force on the first chamber plunger, and the needle deployment is completed, and the drug delivery continues until the required volume of drug is administered.

Once the required volume/dose of the reconstituted drug mixture is administered, the injection process is completed.

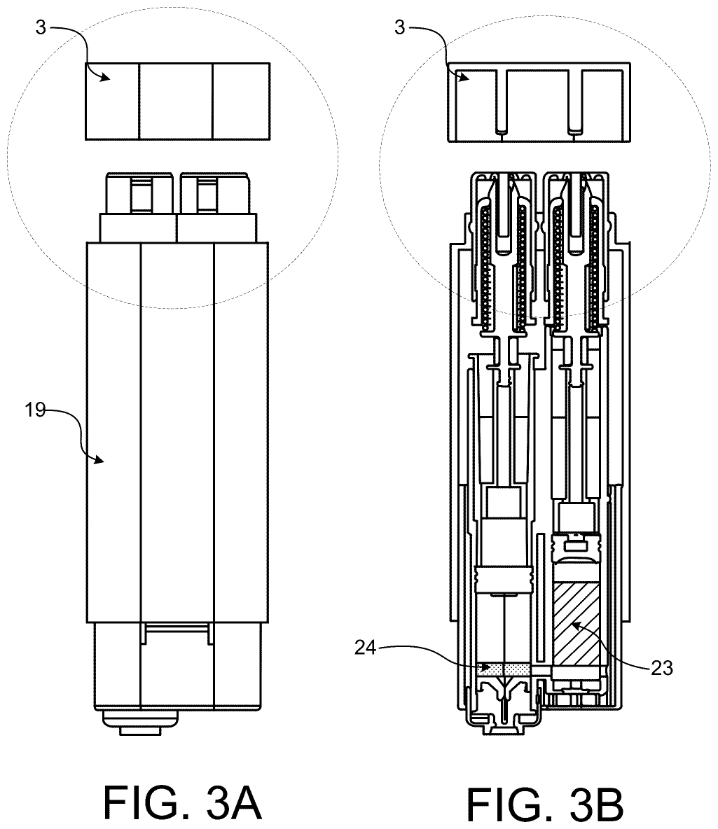

FIGS. 3A-10 illustrate exemplary features of the reconstitution auto-injector during the method for administering a drug using an auto-injector according to embodiments described herein. As shown herein, the method may comprise a safety release portion, an activation portion, a reconstitution portion, an injection portion, and may thereafter be complete. The safety release portion may be optional.

FIGS. 3A-3B illustrate exemplary features of the auto-injector according to embodiments of the invention including a safety feature and the auto-injector during a safety release portion of the method for administering a drug using an auto-injector according to embodiments described herein. The illustration of FIGS. 3A-3B show the safety cap, identified as the safe pin removed from a terminal end of the system, thus separating the cap from the outer body.

In an exemplary embodiment, the reconstitution auto-injector system may be received in packaging. The system may therefore be removed from the packaging. The reconstitution auto-injector may be held with one hand on the outer body 19. The other hand may be used to remove the safety cap 3 from the reconstitution auto-injector, separating the cap from the outer body.

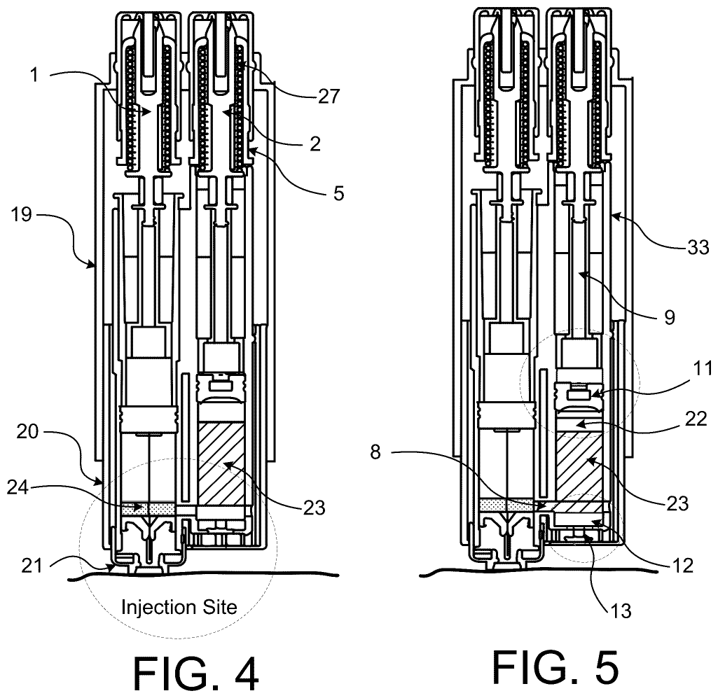

FIG. 4 illustrates a cross sectional view of the auto-injector during an activation portion of the method for administering a drug using the auto-injector according to embodiments described herein.

In an exemplary embodiment of the methods of using the reconstitution auto-injector, when the reconstitution auto-injector is ready for use, the end cap 21 (which may be referred to as a nose of the device) may be positioned to contact the patient. The end of the system may be positioned adjacent the injection site, and the system ready to use.

The method may include pressing the reconstitution auto-injector system against the injection site. As the reconstitution auto-injector is pressed against the patient, as the user is holding the outer body 19, the injector body 33 (an interior portion of the outer body 19) may press against the inner tube 5 and activate the second power source 2. The injector body 33 and the inner body 20 may both move independent of the outer body 19. The power sources 1, 2 are attached to the outer body 19, so the injector body 33 can move towards the terminal end of the reconstitution auto-injector system toward the end with the power sources 1, 2. The injector body 33 may press on the inner tube 5 of the second power source 2. The second power source 2 may be activated first and may be used through the respective plungers to automatically activate the first power source 1 that is activated second. The first activated power source is described herein as the second power source 2 and the second activated power source is described herein as the first power source 1. Therefore, second power source 2 gets activated first to reconstitute the drug, and second power source 1 gets activated after the drug is reconstituted, so that it can inject the drug into the patient. Once the second power source 2 is activated, the second spring 27 is released, and the spring applies a force on the second plunger rod 9.

FIGS. 5-7 illustrates a cross sectional view of the auto-injector during a reconstitution portion of the method for administering a drug using an auto-injector according to embodiments described herein.

As seen in FIG. 5, the method may include moving the second plunger 9 (diluent chamber plunger) in the direction of the injection site, compressing the gas 22 within the second chamber and displacing the liquid 23 (diluent). The stopper 12 gets displaced by the compressed gas 22 and liquid 23, and the plug 13 disengages from the injector body. The stopper 12 then moves to its final position toward the end of the reconstitution auto-injector adjacent the injection site. As the second plunger 11 (diluent chamber plunger) applies force, the plunger causes the liquid 23 (diluent) to flow into the first chamber (identified as the powder chamber) through the flow channel 8.

As seen in FIG. 6, the second plunger 17 (diluent chamber plunger) displaces the liquid 23 (diluent) into the first chamber (powder chamber). The space within the first chamber fills and through the addition of the liquid 23 within the first chamber and/or compression of the gas 22 within the first chamber, a load is applied to the first plunger 17 (powder chamber plunger). As the liquid 23 is injected into the first chamber, the liquid 23 and/or gas 22 within the first chamber move the plunger away from the end cap 21 (or needle). As the liquid 23 enters the first chamber, the diluent starts to reconstituted the powder drug 24.

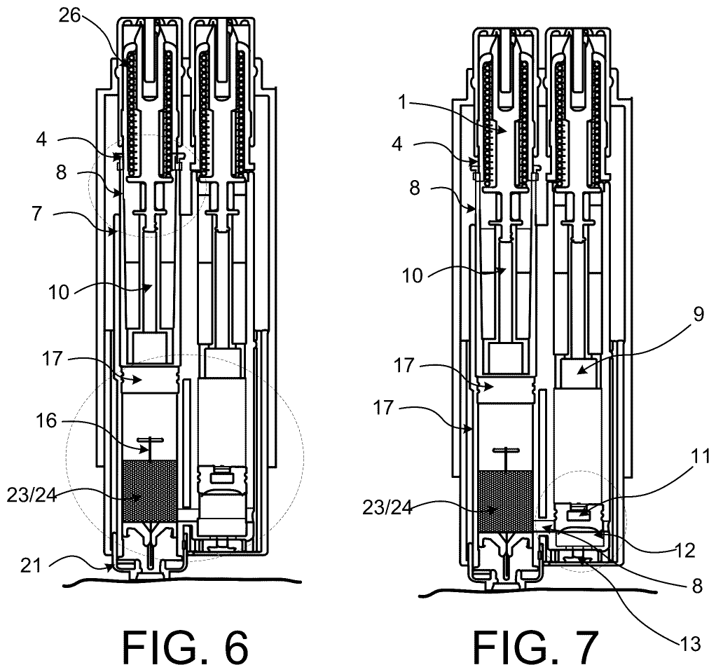

As seen in FIGS. 6 and 7, the second chamber plunger 11 (diluent chamber plunger) displaces the gas within the second chamber (diluent chamber gas/air) into the first chamber (powder chamber), thereby mixing the powder 24 and liquid 23. In an exemplary embodiment of the method described herein, the addition of the gas from the second chamber into the first chamber through the flow channel 8 creates a bubbling into the drug to agitate the drug and liquid to increase the drug reconstitution. As the second plunger 11 reaches the end of the second chamber, the second plunger 11 (diluent chamber plunger) compresses the gas 22 within the liquid/gas 23/24 into the first chamber and is finally positioned adjacent the flow channel 8, thereby closing the flow channel and retaining the fluid/gas 23/24 within the first chamber. In an exemplary embodiment of the method described herein, additional pressure may be created in the first chamber (powder chamber) during the compression of additional gas from the second chamber into the first chamber before the second plunger reaches the end of the second chamber. The compressed gas applies a force on the first plunger 17 (powder chamber plunger) thereby moving the first plunger in the direction of the first power source 1 and away from the end of the system adjacent the injection site (end cap 21). As the first plunger 17 (powder chamber plunger) moves toward the first power source 1, the plunger applies a force and moves the activator 18, which thereafter presses onto the first inner tube 4 and activates the first power source 1. When the first power source 1 is activated, the first spring 26 is released, and elongates. The first spring 26 applies a force on the first plunger rod 10 (Powder chamber Plunger Rod) and therethrough displaces the plunger 17 (Powder Chamber Plunger) in the direction away from the first power source 1 and toward the needle or end cap 21 of the system adjacent the injection site. The first plunger 17 (Powder Chamber Plunger) is moved toward the reconstituted drug mixture 23/24 and compresses the first chamber gas.

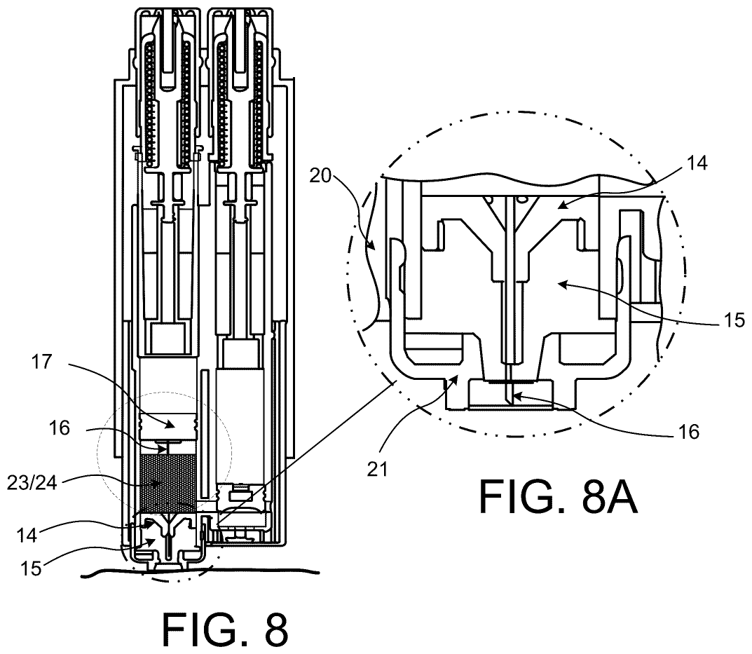

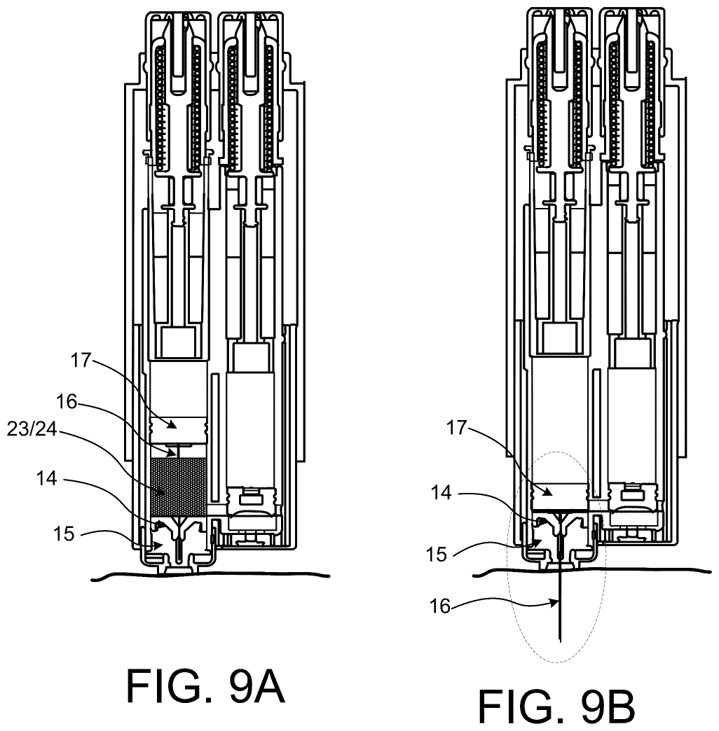

FIGS. 8-9B illustrates a cross sectional view of the reconstitution auto-injector during an injection portion of the method for administering a drug using an auto-injector according to embodiments described herein.

As illustrated in FIGS. 8 and 8A, as the first power source 1 is activated, the first plunger 17 (Powder chamber Plunger) is displaced in the direction of the reconstituted drug mixture 23/24 and compresses the gas within the first chamber and pushes the needle 16 through the stopper 15 (Powder Chamber Stopper). The compressed gas is exhausted after the stopper is punctured in the space between the injection site and the stopper created by the end cap 21.

As illustrated in FIGS. 9A-9B, the first power source 1 and the first plunger 17 (Powder Chamber Plunger) continue to push the needle 16 into the injection site. As the needle 16 is injected into the injection site, the needle moves through the reconstituted drug mixture 23/24. The needle 16 comprises a sharp end for penetrating the patient at the injection site. The opposite end of the needle comprises an orifice on a side of the needle to provide access to the drug for delivery through a hollow passage in the needle from the orifice to an opening at the sharp end of the needle. As the needle is inserted, the reconstituted drug mixture contacts and is able to pass through the needle orifice. The drug is thereafter injected into the injection site as the needle is being deployed into the tissue. The needle deployment and drug delivery are complete as the first plunger 17 is moved to the end of the first chamber.

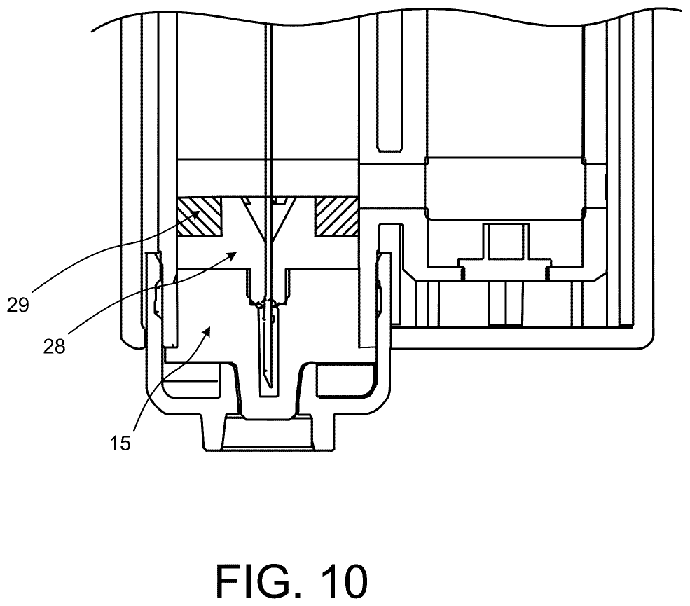

FIG. 10 illustrates a cross sectional view of a portion of the reconstitution auto-injector after completing the method for administering the drug using the auto-injector.

As illustrated in FIG. 10, a reserve drug volume/dose 29 may be available in case the device is activated upside-down or in a compromised drug administration position, and if some amount of drug is squirted out of the system prior to the needle penetrating the injection site. The system may be designed so that the reserve volume is not injected out of the device if the device is activated in the natural position. The reserve volume may be stored in the device's residual volume space, such as within a space of the disc and/or within a volume between the disc and/or the stopper 15 in the first chamber.

As used herein, the terms “about,” “substantially,” or “approximately” for any numerical values, ranges, shapes, distances, relative relationships, etc. indicate a suitable dimensional tolerance that allows the part or collection of components to function for its intended purpose as described herein. Numerical ranges may also be provided herein. Unless otherwise indicated, each range is intended to include the endpoints, and any quantity within the provided range. Therefore, a range of 2-4, includes 2, 3, 4, and any subdivision between 2 and 4, such as 2.1, 2.01, and 2.001. The range also encompasses any combination of ranges, such that 2-4 includes 2-3 and 3-4.

Although embodiments of this invention have been fully described with reference to the accompanying drawings, it is to be noted that various changes and modifications will become apparent to those skilled in the art. Such changes and modifications are to be understood as being included within the scope of embodiments of this invention as defined by the appended claims. Specifically, exemplary components are described herein. Any combination of these components may be used in any combination. For example, any component, feature, step or part may be integrated, separated, sub-divided, removed, duplicated, added, or used in any combination and remain within the scope of the present disclosure. Embodiments are exemplary only, and provide an illustrative combination of features, but are not limited thereto.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

The invention claimed is:

1. A reconstitution auto-injector, comprising a body having:
a first chamber configured to store a powder therein;
a second chamber configured to store a liquid.

2. The reconstitution auto-injector of claim 1, wherein the body comprises an outer body at a first end and an inner body at a second end, the second end comprising an end of the system configured to be positioned adjacent an injection site during use, a portion of the inner body positioned within a portion of the outer body, the first chamber and the second chamber positioned side by side and separated by a separator.

3. The reconstitution auto-injector of claim 2, wherein the first chamber comprises a powder positioned therein, and the second chamber comprises a liquid positioned therein, and the separator comprises a flow channel providing fluid communication between the first chamber and the second chamber, wherein the second chamber comprises a stopper positioned to obstruct the flow channel so that the first chamber and the second chamber are not in fluid communication in a first configuration.

4. The reconstitution auto-injector of claim 3, further comprising a first power source and a second power source, the first power source in communication with a first plunger and configured to move the plunger away from the first power source, and the second power source in communication with a second plunger and configured to move the plunger away from the second power source.

5. The reconstitution auto-injector of claim 4, wherein the second power source is configured to activate upon pressure on the inner body as the system is pressed against an injection site of a patient.

6. The reconstitution auto-injector of claim 5, the second power source comprising a first compressed spring configured to release upon activation and extend to move a second plunger rod and the second plunger away from the second power source and compress a volume of the second chamber comprising the liquid.

7. The reconstitution auto-injector of claim 5, the first power source comprising a second compressed spring configured to release upon activation and extend to move the first plunger rod and the first plunger away from the first power source and compress a volume of the first chamber.

8. The reconstitution auto-injector of claim 7, further comprising an actuator between the first plunger and the first power source configured to release the first spring as the first plunger is moved toward the first power source.

9. The reconstitution auto-injector of claim 1, further comprising a needle, a stopper, and an end cap, wherein the stopper is positioned within the first chamber adjacent the end cap, and the needle is configured to extend into the stopper and to move through the stopper and end cap.

10. The reconstitution auto-injector claim 9, further comprising a safety cap.

11. A method of using a reconstitution auto-injector, comprising:
providing the reconstitution auto-injector;
activating the reconstitution auto-injector;
reconstituting a drug in a powder form into a reconstituted drug mixture; and
injecting the reconstituted drug mixture into a patient.

12. The method of claim 11, wherein the reconstitution auto-injector comprises a powder contained in a first chamber and a liquid contained in a second chamber, wherein the powder and liquid are maintained separate from each other in a first configuration prior to use.

13. The method of claim 12, wherein activating the reconstitution auto-injector comprises pressing the reconstitution auto-injector against a skin of the patient to automatically trigger the reconstitution and the injection.

14. The method of claim 13, wherein the activation comprising releasing a second compressed spring to move a second plunger in a direction away from the second compressed spring and toward an injection site.

15. The method of claim 14, further comprising opening a flow channel between the second chamber and the first chamber so that as the second plunger moves by the second spring, the liquid moves into the first chamber.

16. The method of claim 15, further comprising applying a gas through the flow channel after the liquid from the second chamber moves into the first chamber to agitate the liquid and reconstitute the powder into the liquid creating a reconstituted mixture, and blocking the flow channel with the second plunger.

17. The method of claim 15, further comprising actuating a first compressed spring by moving a first plunger in the first chamber as the first chamber is filled with the liquid from the second chamber, thereby releasing the first compressed spring and applying a force on the first plunger to move the first plunger away from the first spring and toward the injection site.

18. The method of claim 17, further comprising moving a needle within the first chamber with the first plunger through a stopper and out an end cap.

19. The method of claim 18, further comprising dispelling gas contained within the first chamber through the needle after an end of the needle exits the stopper, while the end of the needle is within a gap created by the end cap and before the end of the needle enters a skin of a patient.

20. The method of claim 19, further comprising moving the needle into the patient at the injection site while dispensing the reconstituted mixture into the patient.

21. A reconstitution auto-injector, comprising a body having:
a first chamber configured to store a powder therein;
a first plunger within the first chamber;
a first power source configured to push the first plunger and reduce a volume within the first chamber;
a first actuator configured to trigger the first power source;
a second chamber configured to store a liquid;
a second plunger in the second chamber; and
a second power source configured to push the second plunger and reduce a volume within the second chamber;
a flow passage between the first chamber and the second chamber such that the liquid can flow into the first chamber when the second plunger is moved by the second power source,
wherein the first plunger, first actuator, and first power source are configured such that the activator automatically triggers the first power source when the liquid of the second chamber flows into the first chamber and moves the plunger in a first direction to increase the volume of the first chamber, and once triggered, the first power source pushes the first plunger in an opposite direction to reduce the volume within the first chamber.

22. The reconstitution auto-injector of claim 21, further comprising a stopper configured to cover the flow passage in a first configuration, and uncover the flow passage in a second configuration, wherein the second configuration is configured to occur when the liquid in the second container is compressed by the second plunger after activation of the second power source.

23. A method of using a reconstitution auto-injector, comprising:
providing the reconstitution auto-injector having a first chamber having a powder contained therein, a second chamber having a liquid contained therein, a first power source, and a second power source;
activating the second power source to start the first power source and compress the liquid and flowing the liquid from the second chamber to the first chamber;
reconstituting a drug in a powder form into a reconstituted drug mixture; and
automatically activating the first power source to inject the reconstituted drug mixture into a patient.

24. The method of claim 23, wherein the automatic activation of the first power source is through a movement of a first plunger in a first direction, and the first power source is configured to move the first plunger, once activated, in a second direction opposite the first direction.

25. The method of claim 24, wherein the activation of the second power source is by pressing the reconstitution auto-injector against a skin of a patient to activate the second power source.

26. The method of claim 25, further comprising keeping the first chamber and second chamber out of fluid contact through a stopper over a flow channel, and then permitting fluid flow from the second chamber to the first chamber by moving the stopper from the flow channel.
